(19) United States
(12) Patent Application Publication
(10) Pub. No.: US 2024/0009416 A1
(43) Pub. Date: Jan. 11, 2024

(54) SYSTEMS AND METHODS FOR DETERMINING FEEDBACK TO A USER IN REAL TIME ON A REAL-TIME VIDEO

(71) Applicant: ResMed Pty Ltd, Bella Vista, NSW (AU)

(72) Inventors: Stewart Joseph WAGNER, Bella Vista, NSW (AU); Michael Christopher HOGG, Bella Vista, NSW (AU)

(21) Appl. No.: 18/252,391

(22) PCT Filed: Nov. 16, 2021
(86) PCT No.: PCT/IB2021/060621

§ 371 Date: May 10, 2023

Related U.S. Application Data
(60) Provisional application No. 63/115,997, filed on Nov. 19, 2020.
(60) Provisional application No. 63/143,374, filed on Jan. 29, 2021.

(51) Int. Cl. A61M 16/06; G06V 40/16

(52) U.S. Cl. CPC A61M 16/0605; G06V 40/165

(57) ABSTRACT

A method includes displaying a first real-time video of at least a portion of a face of a user, at least a portion of a head of the user, or a combination of the at least a portion of the face and the at least a portion of the head of the user. The first real-time video is modified by superimposing a virtual user interface. A second real-time video of at least a portion of a face of a user, at least a portion of a head of the user, or a combination of the at least a portion of the face and the at least a portion of the head of the user is displayed. The user in the second real-time video wearing a physical user interface. Feedback is provided to the user in real time on the second real-time video.

### CROSS-REFERENCE TO RELATED APPLICATIONS

This application claims the benefit of, and priority to, U.S. Provisional Patent Application No. 63/115,997 filed on Nov. 19, 2020 and U.S. Provisional Patent Application No. 63/143,374 filed on Jan. 29, 2021, each of which is hereby incorporated by reference herein in its entirety.

### TECHNICAL FIELD

The present disclosure relates generally to systems and methods for determining feedback to a user on a real-time video, and more particularly, to systems and methods for determining feedback to a user in real time on a real-time video relating to a physical user interface.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), chest wall disorders, and insomnia. Many of these disorders can be treated using a respiratory therapy system, while others may be treated using a different technique. These disorders are often diagnosed through a sleep study, and a therapy is prescribed based on the results. However, to obtain more accurate data, the physical user interface in a respiratory therapy system needs to be clinically positioned to provide a desired seal without leakage. The present disclosure is directed to solving these and other problems.

### SUMMARY

According to some implementations of the present disclosure, a method includes displaying a first real-time video of at least a portion of a face of a user, at least a portion of a head of the user, or a combination of the at least a portion of the face and the at least a portion of the head of the user. The method also includes modifying the first real-time video by superimposing a virtual user interface. The method also includes displaying a second real-time video of at least a portion of a face of a user, at least a portion of a head of the user, or a combination of the at least a portion of the face and the at least a portion of the head of the user. The user in the second real-time video is wearing a physical user interface. The method also includes providing feedback to the user in real time on the second real-time video.

According to some implementations of the present disclosure, a system for providing feedback to a user in real-time. The electronic interface is configured to receive (1) a first real-time video of at least a portion of a face of a user, at least a portion of a head of the user, or a combination of the at least a portion of the face and the head of the user; and (2) a second real-time video of at least a portion of a face of a user, at least a portion of a head of the user, or a combination of at least a portion of the face and the head of the user. The user in the second real-time video is wearing a physical user interface. The memory stores machine-readable instructions. The control system includes one or more processors configured to execute the machine-readable instructions to modify the first real-time video by superimposing a virtual user interface, and to provide feedback to the user in real time on the second real-time video.

According to another implementation, a method includes generating image data associated with at least a portion of a face of an individual who has disengaged a physical user interface after a first period of time on the face of the individual. The image data is generated within a second period of time. The generated image data is analyzed to determine one or more characteristics of the face of the individual. Feedback is provided to the individual based at least in part on the determined one or more characteristics of the face of the individual.

According to a further implementation, a method includes engaging a physical user interface to a face of an individual. After a first period of time, the physical user interface is disengaged from the face of the individual. After the disengaging and within a second period of time, image data associated with at least a portion of the face of the individual is generated. The generated image data is analyzed to determine one or more characteristics of the face of the individual. Feedback is provided to the individual based at least in part on the determined one or more characteristics of the face of the individual.

According to a further implementation, a system includes an electronic interface, a memory, and a control system. The electronic interface is configured to receive a generated image data associated with at least a portion of the face of the individual who has disengaged a physical user interface after a first period of time on the face of the individual. The memory stores machine-readable instructions. The control system includes one or more processors configured to execute the machine-readable instructions to: (1) analyze the generated image data to determine one or more characteristics of the face of the individual; and (2) provide feedback to the individual based at least in part on the determined one or more characteristics of the face of the individual.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a user interface of the respiratory system of FIG. 1, according to some implementations of the present disclosure;

FIG. 3B is a perspective exploded view of the user interface of FIG. 3A, according to some implementations of the present disclosure; and

FIG. 4 is a process flow diagram for a method for providing feedback to a user in real time on a real-time video, according to some implementations of the present disclosure.

FIG. 5 is a process flow diagram for a method for providing feedback to a user after generating an image date associated with a portion of a face of an individual who has disengaged a physical user interface, according to some implementations of the present disclosure.

FIG. 6A is a front view of a face before placing a physical user interface thereon;

FIG. 6B is the front view of FIG. 6A within a second period of time after a physical user interface has been removed in one embodiment.

FIG. 7 is a process flow diagram for a method for providing feedback to a user after generating an image date associated with a portion of a face after disengaging a physical user interface, according to another implementation of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

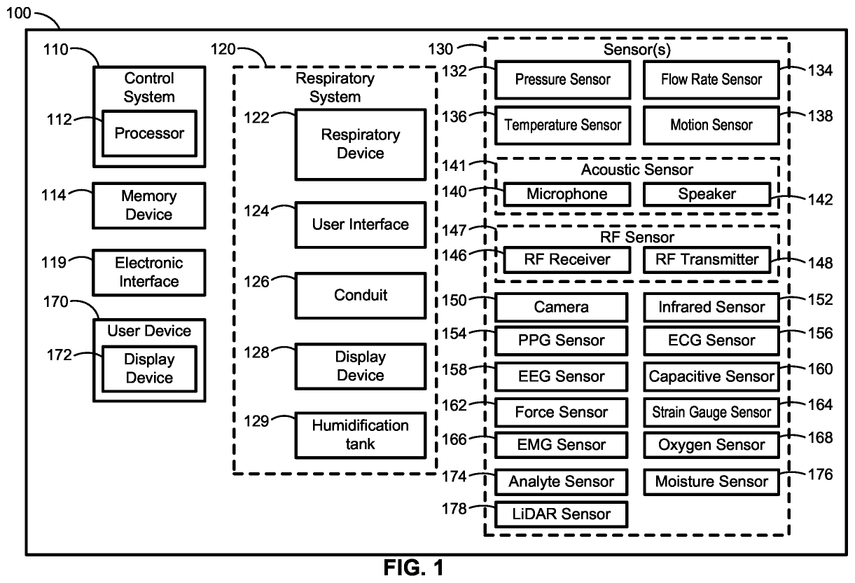

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory system 120, a blood pressure device 180, an activity tracker 190, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, a portion (e.g., a housing) of the respiratory system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

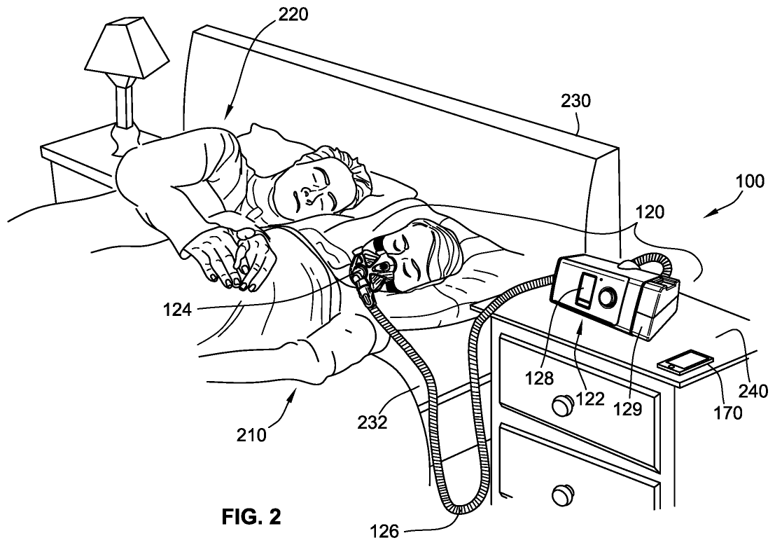

As shown in FIG. 2, in some implementations, the user interface 124 is a full face mask that covers the nose and mouth of the user. Alternatively, the user interface 124 can be a nasal mask that provides air to the nose of the user or a nasal pillow mask that delivers air directly to the nostrils of the user. The user interface 124 may be a tube-up mask, optionally wherein straps of the mask are configured to act as conduit(s) to deliver pressurized air to the face or nasal mask. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210.

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score (also referred to as a myAir score), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory system 120 and a bed partner 220 are located in a bed 230 and are laying on a mattress 232. The user interface 124 (e.g., a full face mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory device 122, the use interface 124, the conduit 126, or the user device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a face mask that covers the nose and mouth of the user 210, the analyte sensor 174 can be positioned within the face mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the face mask (in implementations where the user interface 124 is a face mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and the user device 170. Thus, various systems can be formed using any portion or portions of the components shown and described herein and/or in combination with one or more other components.

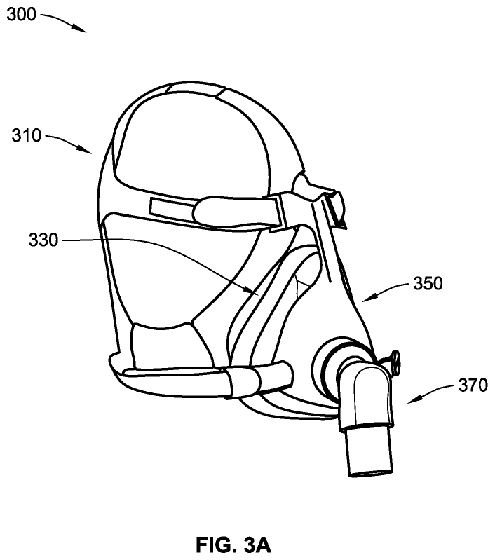

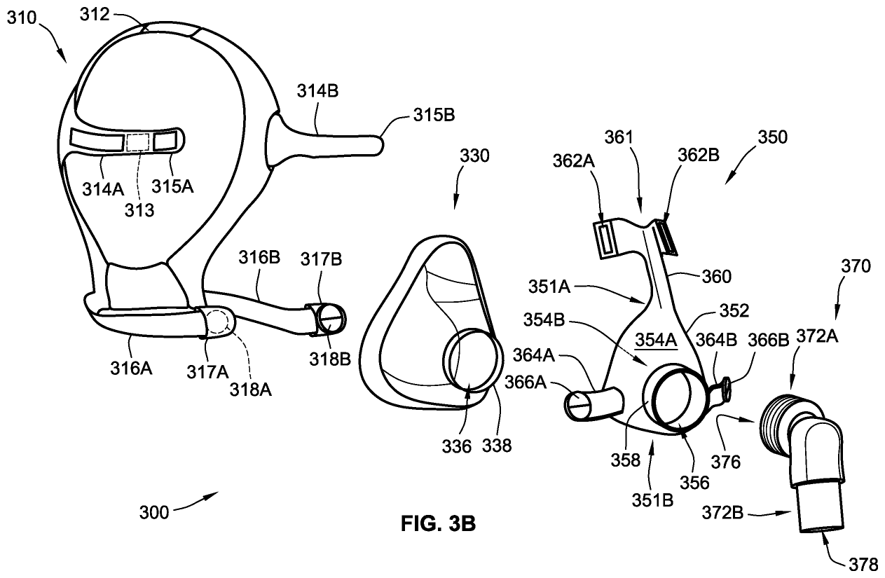

Referring to FIGS. 3A and 3B, a user interface 300, according to some implementations of the present disclosure, is illustrated. The user interface 300 can be the same as, or similar to, the user interface 124 (FIGS. 1 and 2), and can be used with the system 100 described herein. The user interface 300 includes a strap assembly 310, a cushion 330, a frame 350, and a connector 370. The strap assembly 310 is configured to be positioned generally about at least a portion of the user's head when the user wears the user interface 300. The strap assembly 310 can be coupled to the frame 350 and positioned on the user's head such that the user's head is positioned between the strap assembly 310 and the frame 350.

In some implementations, the cushion 330 is positioned between the user's face and the frame 350 to form a seal on the user's face. A first end portion 372A of the connector 370 is coupled to the frame 350, while a second end portion 372B of the connector 370 can be coupled to a conduit (e.g., the conduit 126 shown in FIGS. 1 and 2). In turn, the conduit can be coupled to the air outlet of a respiratory device (e.g., the respiratory device 122 described herein). A blower motor in the respiratory device is operable to flow pressurized air out of the air outlet, to thereby provide pressurized air to the user. The pressurized air can flow from the respiratory device and through the conduit, the connector 370, the frame 350, and the cushion 330, until the air reaches the user's airway through the user's mouth, nose, or both.

The strap assembly 310 is formed from a rear portion 312, a pair of upper straps 314A and 314B, and a pair of lower straps 316A and 316B. The rear portion 312 of the strap assembly is generally positioned behind the user's head when the user wears the user interface 300. The upper straps 314A, 314B and the lower straps 316A, 316B extend from the rear portion 312 toward the front of the user's face. In the illustrated implementation, the rear portion 312 has a circular shape. However, the rear portion 312 may also have other shapes. The rear portion 312, the upper straps 314A, 314B, and the lower straps 316A, 316B can be formed or woven from a generally stretchy or resilient material, such as fabric, elastic, rubber, etc., or any combination of materials. In some implementations, the strap assembly 310 has a hollow interior or channel through which electrical wires or traces may extend, as discussed in further detail below.

The upper straps 314A, 314B and the lower straps 316A, 316B each have first ends originating at the rear portion 312, and second ends that couple to the frame 350. When the user wears the user interface 300, the tension provided by the strap assembly 310 holds the frame 350 to the user's face, thus securing the user interface 300 to the user's head.

In some implementations, a tension sensor can be embedded in one of the straps of the strap assembly. For example, FIG. 3B illustrates a tension sensor 313 embedded in upper strap 314A. The tension sensor 313 is configured to measure tension in the straps of the user interface 124. As discussed, the user interface 124 is generally fasted to the user 210's head using straps that can be tightened using hook and loop fasteners. The tension sensor 313 can sense the tension in the straps, which can then be used to inform and/or instruct the user 210 about the correct fitting of the user interface 124. The tension sensor 313 can be integrated into yarn, fiber, wire, carbon fiber, warps, webs, etc. As the tension in the strap increases or decreases, the sensor element of the tension sensor 313 is deflected, causing a change in the voltage of an output signal. The tension sensor 313 can have high elasticity and low resistance, and the ability to be washed. In some implementations, the tension sensor 313 measures the diameter of an inflatable body by the principles of respiratory inductance plethysmography. The tensor sensor 313 can also be an electric impedance plethysmography sensor, a magnetometer, a strain gauge sensor, or be made of piezo-resistive material displacement sensor.

The frame 350 is generally formed from a body 352 that defines a first surface 354A and a second opposing surface 354B. When the user wears the user interface 300, the first surface 354A faces away from the user's face, while the second surface 354B faces toward the user's face. The frame also defines an annular aperture 356 into which the cushion 330 and the connector 370 can be inserted, to thereby physically couple the cushion 330 and the connector 370 to the frame 350.

The cushion 330 can be coupled to the inside of the frame 350 adjacent to the second surface 354B, such that the cushion 330 is positioned between the user's face and the frame 350. The cushion 330 can be made from the same or similar material as the cushion of user interface 124, for example, a conformal material that aids in forming an air-tight seal with the user's face. The cushion 330 defines an aperture 336, and includes an annular projection 338 extending from the cushion 330 about the aperture 336 of the cushion. The annular projection 338 is inserted into the annular aperture 356 of the frame 350, such that the annular aperture 336 of the cushion 330 overlaps with the annular aperture 356 of the frame 350. In some implementations, the annular projection 338 of the cushion 330 is releasably secured to the body 352 of the frame 350 via a friction fit between the annular projection 338 and the body 352 around the annular aperture 356.

In other implementations, the annular projection 338 and the frame 350 can have mating features that mate with each other to secure the cushion 330 to the frame 350. For example, the annular projection 338 of the cushion 330 may include an outwardly-extending peripheral flange, and the body 352 of the frame 350 can include a corresponding inwardly-extending peripheral flange about the annular aperture 356. When the annular projection 338 of the cushion 330 is inserted into the annular aperture 356 of the frame 350, the peripheral flanges can slide or snap past each other, to thereby secure the cushion 330 to the frame 350. In additional implementations, the cushion 330 is held in place by the tension provided by the strap assembly 310, and is not physically coupled to the frame 350. In still other implementations, the cushion 330 and the frame 350 can be formed as a single integral piece.

The connector 370 can be coupled to the opposite side of the frame 350 in a similar manner to the cushion 330. The first end portion 372A of the connector 370 has a generally cylindrical shape and can be inserted into the annular aperture 356 of the frame 350, such that a hollow interior 376 of the end portion 372A overlaps with the annular aperture 356, and the aperture 336 of the cushion 330. The opposing second end portion 372B of the connector 370 is then coupled to the conduit, such that the user's face (including the user's mouth and/or nose) is in fluid communication with the conduit through the cushion 330, the frame 350, and the connector 370.

The first end portion 372A of the connector 370 is generally annular-shaped, and fits into the annular aperture 356 of the frame 350. The frame 350 also includes an annular projection 358 that extends from the second surface 354B of the frame 350 and is formed about the annular aperture 356. When the first end portion 372A is inserted into the annular aperture 356 of the frame 350, an inner surface of the annular projection 358 overlaps with an outer surface of the first end portion 372AA of the connector 370.

In some implementations, a friction fit between the annular projection 358 and the first end portion 372A secures the connector 370 to the frame 350. In other implementations, the connector 370 can include a fastener configured to secure the connector 370 to the frame 350 (e.g., via a threaded connection). In one example, the annular projection 358 has an outwardly-extending peripheral flange, and the fastener is one or more deflectable latches formed on the first end portion 372A of the connector 370. As the first end portion 372A slides is inserted within the annular projection 358, the deflectable latch slides over the peripheral flange such that the deflectable latch is positioned outside of the annular projection 358. As the deflectable latch passes by the peripheral flange, the peripheral flange pushes the deflectable latch away from the annular projection 358. The deflectable latch then returns to its original position, such that the connector 370 cannot be removed from the frame 350 without manually deflecting the deflectable latch away from the annular projection 358.

The frame 350 includes a T-shaped extension strip 360 extending upward from an upper end 351A of the body 352. In some implementations, the extension strip 360 is integrally formed with the body 352. In other implementations, the extension strip 360 is a separate component that is coupled to the body 352. When the user wears the user interface 300, the extension strip 360 generally extends up to the user's forehead. In some implementations, the extension strip 360 includes a cooling portion or mechanism that contacts and cools the user 210's forehead, which can help users with insomnia fall asleep.

The lower straps 316A, 316B extend toward the frame 350 from the rear portion 312 of the strap assembly 310, and are coupled to opposite sides of a lower end 351B of the body 352. The upper straps 314A, 314B extend toward the frame 350 from the rear portion 312 of the strap assembly 310, and are coupled to opposite sides of the upper end 361 extension strip 360 (e.g., the generally horizontal “cross” of the T). The frame 350 can include a variety of different strap attachment points to couple with the upper straps 314A, 314B and the lower straps 316A, 316B.

One type of strap attachment point is shown in the extension strip 360. The upper end 361 of the extension strip 360 includes two apertures 362A, 362B. These apertures can be integrally formed in the extension strip 360 itself, or may be formed as part of a separate component or piece that is coupled to the extension strip 360. The apertures 362A, 362B are shaped to allow the ends 315A, 315B of the upper straps 314A, 314B to be inserted through the apertures 362A, 362B. The ends 315A, 315B can then loop back and fasten to remainder of the upper straps 314A, 314B via any suitable mechanism, such as hook and loop fasteners, adhesive, etc. The upper straps 314A, 314B are thus secured to the extension strip 360 of the frame 350.

The frame 350 is shown with a different type of strap attachment point used to couple the lower straps 316A, 316B to the frame 350. The frame 350 includes two lateral strips 364A, 364B extending away from opposite ends of the lower end 351B of the body 352. The first end of each lateral strip 364A, 364B is coupled to the body 352, and a corresponding magnet 366A, 366B is disposed at the second end of each lateral strip 364A, 364B. A magnet 318A is coupled to end 317A of lower strap 316A, while a magnet 318B is coupled to end 317B of lower strap 316B. Magnet 318A can be secured to magnet 366A via magnetic attraction, while magnet 318B can be secured to magnet 366B via magnetic attraction, to thereby couple the lower straps 316A, 316B to the body 352 of the frame 350.

In some implementations, the frame 350 does not include the extension strip 360, and the upper straps 314A, 314B are instead coupled to the frame, above the lateral strips 364A, 364B. The upper straps 314A, 314B in these implementations extend past the user 210's temples and around to the rear of the user 210's head. The frame 350 may include upper lateral strips which the upper straps 314A, 314B are coupled to.

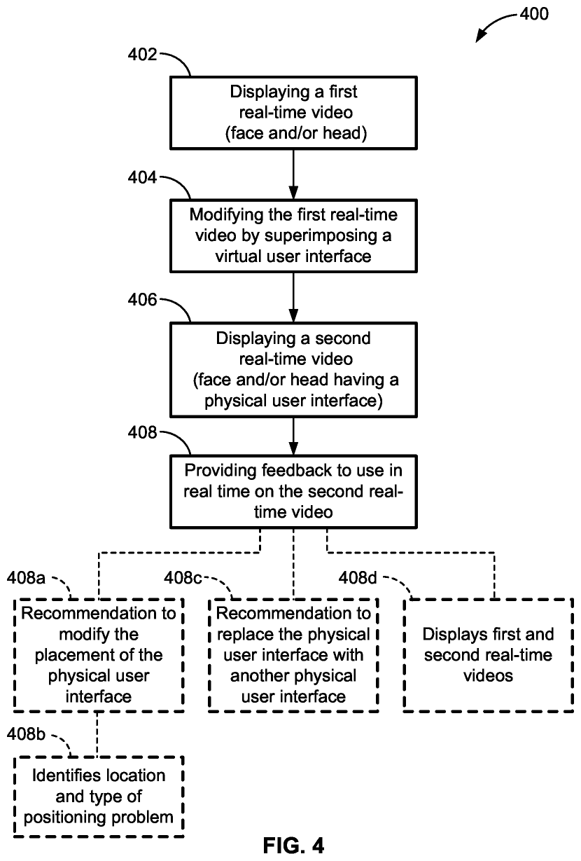

Referring to FIG. 4, a method 400 for providing feedback to the user in real time on a real-time video is illustrated. Some individuals have a difficult time, at least initially, of properly placing a physical user interface onto a user's face/head despite instructions on how to do so. This feedback will assist a user in properly fitting and positioning the physical user interface. As a result, leaks from the physical user interface being located improperly (e.g., the user interface being located upside down or not being strapped properly) are prevented or inhibited. One or more steps or aspects of the method 400 can be implemented using any portion or aspect of the system 100 described herein.

Step 402 of the method 400 includes displaying a first real-time video of at least a portion of a face of a user, at least a portion of a head of the user, or a combination of the at least a portion of the face and the at least a portion of the head of the user. The first real-time video in one embodiment may be generated by a user using a camera. The camera may be a traditional camera that takes the first real-time video. The camera is often a camera located in a user device. For example, the camera 150 may be contained within the user device 170. The user device 170 may be a smart phone, a tablet, a laptop or other device described above having a camera. The display may be a display 172 described above.

The camera 150 may be a two-dimensional camera or a three-dimensional camera. In a two-dimensional camera embodiment, it is noted that the scaling of the face can be accomplished by a fixed item of known size (e.g., a coin) or by using a fixed feature in a user's face (e.g., the size of an iris). Scaling can occur where a partial view of a face and/or head is taken in a first real-time video. The two-dimensional camera may be a two-dimensional camera with a depth sensor (e.g., a dot projector and infrared sensor). Such cameras may be found, for example, in smartphones with front-facing cameras. The camera is not limited to being a front-facing camera, but may be easier to see feedback on the display in real time. The camera be a rear-facing camera in which the lens faces away from the user, just like in a regular digital camera.

Step 404 of the method 400 includes modifying the first real-time video by superimposing a virtual user interface. This is referred to as augmented reality. The selection of the virtual user interface may be manually inputted or automatically inputted as will be discussed below. An automated selection is more likely used when the user does not have a physical user interface, while a manual selection is more likely used when the user has a physical user interface or has identified a particular physical user interface to be used

In one embodiment, the virtual user interface is a mask. The mask to be used as the virtual user interface may be, for example, a full face mask, a nasal mask, or a nasal pillow mask. For example, the virtual user interface may be created from images such as shown with respect to the user interface 124 (FIG. 2) or the user interface 300 (FIGS. 3A and 3B) described above. It is desirable to have multiple types and sizes (e.g., small, medium, large, extra-large, etc.) of virtual user interfaces to correspond with a variety of user's faces and heads. By having multiple types and sizes, a user may obtain a better feel for what a properly selected physical user interface should look like when placed on the face/head.

In one method, the superimposed virtual user interface desirably moves in sync in the first real-time video with at least a portion of a face of a user, at least a portion of a head of the user, or a combination of the at least a portion of the face and the head of the user. Thus, if the user moves his or her face to the left, the superimposed virtual user interface will move in sync to the left to match the movement of the face. Thus, a user will be able to view the superimposed virtual user interface properly positioned with respect to any position of the user's face/head. It is also contemplated that the superimposed virtual user interface may move in sync with specific features in the first real-time video.

In another method, an input is provided for the user to select the virtual user interface to be superimposed in the first real-time video. In this method, the user selects the type of virtual user interface that generally corresponds to the actual physical user interface to be worn by the user. This selection should assist a user on how the physical user interface should be placed and positioned on the user's face/head. This can also assist a user in viewing how other types of user interfaces may look ON the user's face/head. The user may select the virtual user interface from a user interface on a scroll-down menu in one method. The user may also select the virtual user interface by scanning a particular mask to be used in the first real-time video. In another method, the user may identify the mask by product name or identification number. In a further method, the user may include measurements of the face and/or head to determine which virtual user interface to select. In yet another method, the user may be asked to fill out a questionnaire to assist in selecting the virtual user interface. It is contemplated that there are other methods for selecting the virtual user interface to be superimposed in the first real-time video.

In some implementations, step 404 includes the virtual user interface may be automatically inputted. In one embodiment, step 404 includes using a trained machine learning algorithm to modify the real-time video by superimposing the virtual user interface. The machine learning algorithm can include, for example, neural networks, convolution neural networks, deconvolution neural networks, recurrent neural networks, generative adversarial networks, or any combination thereof. The machine learning algorithm can be trained (e.g., using supervised or unsupervised training techniques) using selected data as an input and outputting the superimposed virtual user interface. That is, the machine learning algorithm can be trained such that it receives selected data for a particular user and outputs a recommended virtual user interface based on the inputted information.

The data that may be used includes the following: (i) information from the first real-time video; (2) database information of a plurality of types and/or sizes of virtual user interfaces; (3) database information of different user's faces/heads; (4) user-entered information; and (5) any combination thereof. The selection of a specific one of the different user's faces/heads in the database may correlate or match up with one or more features of the user in the first real-time video. In other words, the machine learning algorithm can use or select from a database of previously stored faces/heads scans to select an appropriately fitting virtual user interface to superimpose in the first real-time video. The machine learning algorithm can learn from the shape of the faces/heads so when there is a partial face/head scan, the rest of the head can be reliably predicted from this database of previously stored faces/heads scans. The machine learning algorithm can then apply such information to the user in the first real-time video.

The user-entered information may include: (1) measurements of the face or the head; (2) measurements of one or more features on the face or head; (3) identification of breathing method; (4) gender; (5) ethnicity; (6) amount of hair; or (7) any combination thereof. The measurements can assist in determining which virtual user interface should be selected and superimposed in the first real-time video. The measurements of the face, head or other feature assist in giving a user's face/head shape can assist in providing a recommendation based on the user's face/head shape. The user's face/head shape can also assist in conduit sizing. In another method, the identification of the breathing method may assist in determining which virtual user interface should be selected and superimposed. For example, if a user is a mouth breather, then the selected virtual user interface may be a full face mask. If a user is a nose breather, then the selected virtual user interface may be a nasal mask.

Step 406 of the method 400 includes displaying a second real-time video of at least a portion of a face of a user, at least a portion of a head of the user, or a combination of the at least a portion of the face and the at least a portion of the head of the user. The second real-time video may be taken or displayed on the same user devices as discussed above with the first real-time video. It is contemplated that the second real-time video may be taken on a different user device than used in the first real-time video.

The second real-time video shows a user with a physical user interface. The physical user interface is a user interface that is physically located on the user. This, of course, differs from a virtual user interface that is superimposed in the first real-time video.

In one embodiment, the physical user interface is a mask. The physical user interface may be, for example, a full face mask, a nasal mask, or a nasal pillow mask. For example, the physical user interface may be the user interface 124 (FIG. 2) or the user interface 300 (FIGS. 3A and 3B) described above.

Step 408 of the method 400 includes providing feedback to the user in real time on the second real-time video. There are various types of feedback to the user that can be provided. Some are these are illustrated in FIG. 4. In one method, the feedback is a recommendation to modify the placement of the physical user interface in the second real-time video is shown in step 408a. In this method, it is determined that placement of the physical user interface is not in the clinically optimal position. As shown in step 408b, this feedback may further include feedback that identifies the location and type of problem with the positioning of the physical user interface in the second real-time video. The feedback may relate to one or more of the following features of a physical user interface: strap assembly, cushion or the connector. The user interface may be the user interface 300 that includes the strap assembly 310, the cushion 330 and the connector 370 discussed above with respect to FIGS. 3A, 3B. This feedback to one or more of the following features may include the following: tightening/loosening strap assembly, relocating the strap assembly, relocating the cushion, reconnecting the connector, or combinations thereof. The positioning is the desired position from a clinical standpoint that is typically related to one or more features of a user.

The feedback may be showing the second real-time video in which a virtual user interface overlays the physical user interface in real-time. In this video, the virtual user interface would indicate the optimal location of the physical user interface. The real-time feedback may be provided when the user is wearing the physical user interface by displaying the ideal location of the physical user interface (e.g., mask). This would optimally include the location of the rigid portion of the physical user interface, as well as the headgear (e.g., straps, etc.) of the physical user interface.

The feedback in step 408 to a user may be audible, visual or a combination thereof. The feedback will typically be displayed on the display device (e.g., display device 172). The visual feedback may be in a written form in one embodiment. In another embodiment, the visual feedback may be a symbol or representation (e.g., an arrow pointing to the feedback or problem area). In a further embodiment, the visual feedback may be a combination of a symbol and text.

The feedback in another method shown in step 408c may be a recommendation to replace the physical user interface in the second real-time video with another physical user interface. This may be of a different type or size of physical user interface. This feedback indicates to a user that the currently used physical user interface in the second real-time video may not be a clinically optimally. By replacing the currently used physical user interface in the second real-time video, the fit will desirably be improved. For example, some physical user interfaces are better fits for certain types of face configuration (e.g., a wider face, a thinner face, thinner nose, nose shape/size).

The feedback in another method displays both the first real-time video and the second real-time video in adjacent sections of a screen. By displaying the first real-time video and the second real-time video in adjacent sections of a user display enables the user to more easily see if the user interface is properly positioned. It is contemplated that the first real-time video and the second real-time video may be located in a side-by-side orientation or a top-to-bottom orientation.

In another embodiment, the real-time feedback may be provided or shown with the user wearing the physical user interface in a first real-time video in combination with displaying the ideal or optimum location of the user interface. This visual feedback may be displayed by superimposing a virtual user interface having its ideal location onto the first real-time video.

To improve clarity, the ideal or optimum location of the physical user interface (including the rigid portion and the headgear) may be shown as dashed or dotted lines on the first-real time video. In another embodiment, certain regions of the first real-time video may be highlighted where the physical user interface and/or conduit is located outside the optimal positions such that the fit may be improved. This highlighted area of the physical user interface could include the rigid portion and the headgear (e.g., straps, etc.). This could be color coded (e.g., a red color) in the area the needs improvement. Additionally, or separately, in another embodiment, the optimal area where the physical user interface should be located is highlighted. This highlighted area of the physical user interface could include the rigid portion and the headgear (e.g., straps, etc.). This could be color coded (e.g., a green color) for the location of the optimal position.

In other embodiments, visual indicators may be used to improve tracking of the virtual user interface when a user moves or switches positions. Some non-limiting examples of visual indicators include elastic indicators, dot indicators, and infrared markers. It is contemplated that other visual indicators may be used.

In one embodiment, elastic indicators are used as visual indicators. The elastic indicator may be placed on a physical user interface such as on the headgear. The elastic indicators may be a plurality of indicators. The plurality of elastic indicators may be located a pre-determined length apart from each other in one embodiment. The elastic indicators may stretch when headgear (e.g., straps) is worn. The post processing of images will allow the stretch of headgear to be determined. This information may be used to determine if the headgear is under/over tightened and, if so, feedback may be provided to the user to tighten/loosen headgear to an appropriate level of tightness for optimum comfort. To assist improving or ensuring accuracy of the elongation or stretching measurements, scaling may be used. If an image is not scaled correctly, the error may be greater than the sensitivity of the elongation measurements of the elastic indicators. Scaling indicia within the image may be used in embodiments. These scaling indicia include, but are not limited to, the diameter of the iris, a coin, or a sizing card.

In another embodiment, dot indicators may be used as visual indicators. The position of the plurality of dot indicators may be tracked on headgear with respect to the head of the user. The dot indicators are used to determine if the headgear and the remainder of the physical user interface correspond to a desired placement of optimal comfort. In one embodiment using a bifurcated headgear strap, the split angle of the upper and lower straps can be estimated, as well as whether placement of the lower and upper straps corresponds to the most stable position on the user's head. To assist in improving the accuracy of the measurements, the angle of the camera to the face is desirably perpendicular. As discussed above scaling indicia, (e.g., a sizing card) may be held up to the face to ensure that there is a reference length/angle between images that is constant.

In another embodiment, infrared markers may be used as visual indicators. Infrared sensors on phones are becoming increasingly common. Infrared markers are easier to select, which assist in ensuring accurate tracking without needing post-processing images. The material to be used would be that would be reflective to infrared light. It can be desirable for the infrared markers to comprise highly reflective material. Some non-limiting examples of reflective material may include aluminum or copper. It is contemplated that other materials may be used as material that are reflective to infrared light. The infrared markers may be used to track position of the physical user interface (e.g., the rigid portion) during sleep, which may provide stimuli/feedback to treat positional apnea such as, for example, determining whether a user is sleeping on his or her side.

In another method, the steps of placing the virtual user interface onto the face and the head of the user may be shown. This will assist the user in properly placing the user interface onto the face and the head of a user. This may also be a shown with a side-by-side orientation with the second real-time video. This will assist the user in following the steps of properly placing the physical user interface onto the face and the head of a user.

These steps of placing the virtual user interface may include moving the cushion into place and then connecting/adjusting the straps. One non-limiting example of a user interface that may be used is user interface 300 described above in FIGS. 3A, 3B. The method may include the use of animated hands placing the virtual user interface onto the face or head of a user. This method may include tips or suggestions to improve the placement of the user interface and consequently the sealing of the same.

To more accurately estimate the optimum area of conduit on a user's head, an estimate of the entire head's shape may be obtained using a 3-dimensional model. The entire head's shape includes the back and the top of the head, as well as the front face. This 3-dimensional model will enable more accurate feedback of where the headgear straps should be positioned on the head (behind the head and around the occipital), as well as determining the optimum force vector angles for the headgear.

In one method, a three dimensional model of the entire head is based on a plurality of two dimensional images of the user taken at various angles and morphing/fitting this model to a statistical shape model. In another method, a three dimensional scanner or depth sensor may be used to obtain a three dimensional model of a user's head (e.g., Iphone's TrueDepth camera). Each of the modeling techniques may obtain (1) one of the user without wearing the physical user interface (e.g., mask); and (2) one of the user wearing the physical user interface. These will assist in determining which areas of the physical user interface do not have an optimum fit.

The phrase “real-time” refers to the actual or substantially actual time in which an event occurs. For example, when a user is taking a video and watching it on a screen (whether that video is being recorded or not), this is a real-time video. It is noted that the image and/or video data associated with a real-time video is being processed so there will or may be some delay associated with the real-time video. For example, in some implementations, depending on the hardware (e.g., smart phone) being used to create and/or display the real-time video, the delay can be measured in nanoseconds, microseconds, milliseconds, seconds, etc. When delay becomes greater than, for example, 30 seconds, such delay can be considered to cause the video to not be real-time.

The phase “real-time video” can refer to a live stream and/or a substantially live stream or play of a video that is recorded or not recorded. By recorded it is meant that the video is stored in memory, which can be temporary memory (e.g., RAM memory) and/or more long term or permanent memory (e.g., solid state hard drives, hard disk drives, etc.) of a device. The long term storage of a video is not necessary, but could be included in one or more implementations of the present disclosure.

In another method, image data associated with at least a portion of a face of an individual after disengagement of a physical user interface. The image data is generated within a second period of time. The generated image data is analyzed to determine one or more characteristics of the face of the individual. Feedback is provided to the individual based at least in part on the determined one or more characteristics of the face of the individual.

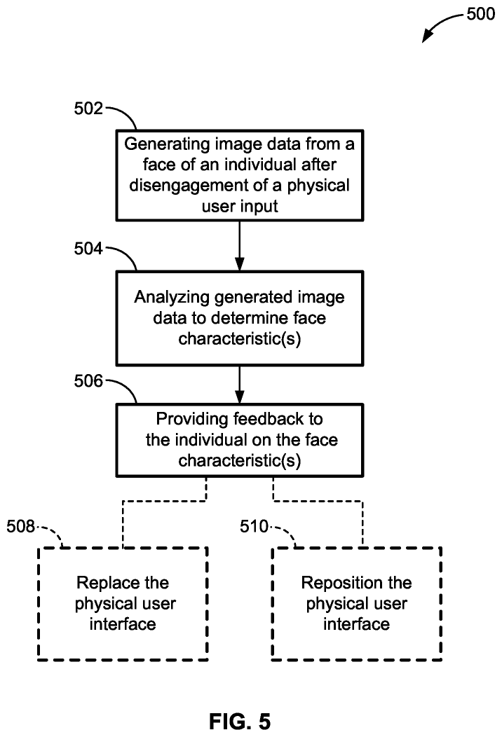

Referring to FIG. 5, a method 500 for providing feedback to an individual on one or more face characteristics of an individual is shown. In step 502 of the method 500, image data from a face of an individual after disengagement of a physical user interface is generated. The physical user interface will typically cover a portion of the face of the individual. One non-limiting example is shown in FIG. 2 with the physical user interface 124. Other non-limiting examples are shown in FIGS. 3A and 3B with the physical user interface 300. The generated image data is reproducible as, for example, a real-time video, a photograph, or both.

The image data is generated within a second period of time. It is desirable for the generated image data to determine one or more characteristics of the face of the individual within a shorter period of time. For example, the second period of time is typically within about 15 or about 30 seconds after disengagement of the physical user interface. The second period of time in another embodiment can be within about 1 or about 2 minutes after disengagement of the physical user interface. It is contemplated that the second period of time may be within 5 minutes or within about 60 minutes after disengagement of the physical user interface.

In step 504 of the method 500, the generated image data is analyzed to determine one or more face characteristics. The analyzing of the generated image data to determine one or more characteristics of the face of the individual in one embodiment includes determining whether there is at least one facial marking on the face of the individual. And, if so, (i) determining the location of the at least one facial marking, and (ii) estimating a depth of at least one facial marking. Information is analyzed from the at least one facial marking on the face of the individual.

After a duration of time, facial markings may form on a portion of the face after disengagement of the physical user interface. The facial markings are typically in the form of a color difference (e.g., redness or other hue) on a portion of the face. The color difference may be on the skin where a portion of the physical user interface was engaged thereon or on a surrounding area where the physical user interface was engaged. The facial markings on a portion of the face are typically the result of the physical user interface being too tight on the face of the user. The facial marking of the face of an individual may also be based on that person's individual anatomy, such as larger features (e.g., cheeks or nose). The facial markings may also be a sign of sensitive skin on certain parts of the face (e.g., the upper lip). Sensitive skin can exacerbate the facial markings from the physical user interface.

The information to be analyzed may include: (1) physical user interface information and/or headgear-related information causing the at least one facial marking; (2) the depth of the at least one facial marking; (3) the length, area and/or shape of the at least one facial marking; (4) the color of the at least one facial marking; and (5) the location of the at least one facial marking relative to one or more features of the face. The color could be a gradient or varying color within the facial marking that could indicate overtightening or ill fit in a certain direction.

The physical user interface information can include the presence of a physical user interface, the type of physical user interface, the size of the physical user interface, or dimensions of the physical user interface.

The depth of the at least one facial marking is determined by a depth sensor in one embodiment. The depth sensor to be used in determining the at least one facial marking may be in, for example, a camera in a smart phone. The estimation of the depth of the at least facial marking may be performed by a three-dimensional camera.

In another method, the depth of the at least one facial marking is determined by a trained machine learning algorithm. The trained machine learning algorithm may use information such as, for example, the color of the at least one facial marking to determine the depth of the at least one facial marking. The trained machine learning algorithm may use information such as, for example, a shadow adjacent to the at least one facial marking to determine the depth of the at least one facial marking. The trained machine learning algorithm may use a combination of the color of the at least one facial marking and the shadow adjacent to the at least one facial marking to determine the depth of the at least one facial marking.

The facial markings on a portion of the face are typically the result of the physical user interface being too tight on the face of the user. The facial markings on a portion of the face may be from an incorrect size of patient interface. The facial marking of the face of an individual may also be based on that person's individual anatomy, such as larger features (e.g., cheeks or nose). It is contemplated that non-optimal selection of the type of physical user interface may be a cause of facial markings on a portion of the face. For example, a cradle cushion may fit better for users with certain anatomical features as compared to a pillow cushion. Machine learning may be used to recommend a better suited or more appropriate mask.

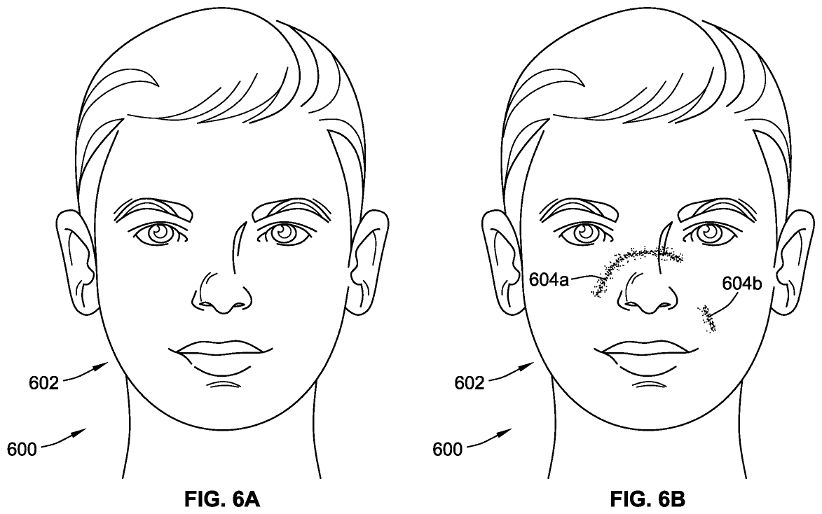

In one non-limiting example shown in FIGS. 6A, 6B, an individual 600 is shown. The individual 600 in FIG. 6A is shown before a physical user interface is placed on a face 602 of the individual 600. FIG. 6B is shown after a physical user interface has been removed from the face 602 of the individual 600. The face 602 in FIG. 6B includes facial markings such as redness areas 604, 604b thereon. The redness areas 604a, 604b occur in areas where the physical user interface previously contacted the face. The redness areas may also occur in areas adjacent to where the physical user interface contacted the face.

Referring back to FIG. 5, in step 506, feedback is provided to the individual based at least in part on the one or more face characteristics of the individual. One recommendation may be to replace the physical user interface such as shown in step 508. More specifically, the provided feedback in one method is a recommendation to replace a worn-out cushion of the physical user interface. The feedback may be a recommendation to change the material forming the seal of a physical user interface (e.g., changing to a silicon cushion for an improved comfort due to an increased compliance of the cushion as compared to foam or a textile). The feedback may be a recommendation for swapping to a different type of physical user interface. For example, a full face mask may be swapped to an ultra-compact (e.g., smaller) full face mask, or a nasal mask may be swapped to a pillow mask/cradle. The feedback may be to size up or size down the cushion if facial markings indicate that the physical user interface is not fitting properly.

A further recommendation may be to reposition the physical user interface such as shown in step 510. The repositioning of the physical user interface may be specific instructions relative to the face of the individual. The recommendation could be to readjust the non-optimal headgear by its angle to have a high or lower force vector. The recommendation may be to loosening certain straps that are overtightened. It is contemplated that other feedback may be provided to the individual based at least in part on the one or more face characteristics of the individual. The timing of this feedback to optimize the fitting of the physical user interface may be provided before a user goes to sleep.

In one method, the provided feedback uses a machine learning algorithm that is trained to receive as an input: (i) information from the generated image data; (ii) information from a database of a plurality of individuals with one or more characteristics of the face thereof; (iii) user-entered information or (iv) any combination thereof.

The user-entered information may be subjective information entered by the user in one method. Some non-limiting examples of user-entered information include the following: (i) how the pressure of the physical user interface feels, (ii) identifying one or more locations of where the pressure of the physical user interface does not feel right, or (iii) a combination thereof. It is contemplated that other user-entered information may be entered by the user.

In a further method, a physical user interface is engaged to a face of an individual. After a first period of time, the physical user interface is disengaged from the face of the individual. After disengaging and within a second period of time, image data associated with at least a portion of a face of the individual is generated. The image data is generated within a second period of time. The generated image data is analyzed to determine one or more characteristics of the face of the individual. Feedback is provided to the individual based at least in part on the determined one or more characteristics of the face of the individual.

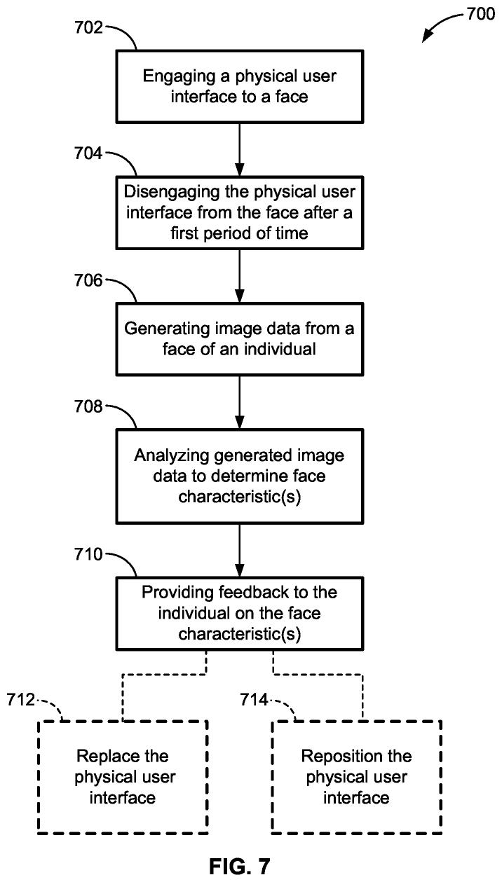

Referring to FIG. 7, a method 700 for providing feedback to an individual on one or more face characteristics of an individual is shown. In step 702 of the method 700, a physical user interface is engaged to a face of an individual. The physical user interface will typically cover a portion of the face of the individual. One non-limiting example is shown in FIG. 2 with the physical user interface 124. Other non-limiting examples are shown in FIGS. 3A and 3B with the physical user interface 300. The generated image data is reproducible as, for example, a real-time video, a photograph, or both.

In step 704 of the method 700, the physical user interface is disengaged from the face of the individual after a first period of time. The first period of time in which the physical user interface is on the face of the individual is generally at least about 2 or about 3 hours. More typically, the first period of time is at least about 5 or about 6 hours. In one method, the first period of time may be based on a sleep period such as the amount of time spent sleeping at night. This first period of time can be at least about 7 or about 8 hours in such a method.

In step 706 of the method 700, image data from a face of an individual after disengagement of the physical user interface is generated within a second period of time. It is desirable for the generated image data to determine one or more characteristics of the face of the individual within a shorter period of time. For example, the second period of time is typically within about 15 or about 30 seconds after disengagement of the physical user interface. The second period of time in another embodiment can be within about 1 or about 2 minutes after disengagement of the physical user interface. It is contemplated that the second period of time may be within 5 minutes or within about 60 minutes after disengagement of the physical user interface.

In step 708 of the method 700, generated image data is analyzed to determine one or more face characteristics. The analyzing of the generated image data to determine one or more characteristics of the face of the individual in one embodiment includes determining whether there is at least one facial marking on the face of the individual. And, if so, (i) determining the location of the at least one facial marking, and (ii) estimating a depth of the at least one facial marking. Information is analyzed from the at least one facial marking on the face of the individual.

The information to be analyzed may include: (1) physical user interface information and/or headgear-related information causing the at least one facial marking; (2) the depth of the at least one facial marking; (3) the length, area and/or shape of the at least one facial marking; (4) the color of the at least one facial marking; and (5) the location of the at least one facial marking relative to one or more features of the face. The color could be a gradient or varying color within the facial marking that could indicate overtightening or ill fit in a certain direction.

In step 710, feedback is provided to the individual on the one or more face characteristics. One recommendation may be to replace the physical user interface such as shown in step 712. More specifically, the provided feedback in one method is a recommendation to replace a worn-out cushion of the physical user interface. The feedback may be a recommendation to change the material forming the seal of a physical user interface (e.g., changing to a silicon cushion for an improved comfort due to an increased compliance of the cushion as compared to foam or a textile). The feedback may be a recommendation for swapping to a different type of physical user interface. For example, a full face mask may be swapped to an ultra-compact (e.g., smaller) full face mask, or a nasal mask may be swapped to a pillow mask/cradle. The feedback may be to size up or size down the cushion if facial markings indicate that the physical user interface is not fitting properly.

A further recommendation may be to replace the physical user interface such as shown in step 714. The repositioning of the physical user interface may be specific instructions relative to the face of the individual. The recommendation could be to readjust the non-optimal headgear by its angle to have a high or lower force vector. The recommendation may be to loosening certain straps that are overtightened. It is contemplated that other feedback may be provided to the individual based at least in part on the one or more face characteristics of the individual. The timing of this feedback to optimize the fitting of the physical user interface may be provided before a user goes to sleep.

In one method, the provided feedback uses a machine learning algorithm that is trained to receive as an input: (i) information from the generated image data; (ii) information from a database of a plurality of individuals with one or more characteristics of the face thereof; (iii) user-entered information or (iv) any combination thereof.

In another method, a further step may include determining whether the physical user interface on the face of the individual is leaking before disengagement from the face of the individual. In this method, the provided feedback to the individual would further include whether the physical user interface is leaking. This may be combined with other methods discussed above, including the methods 500 and 700.

When there is space between the face and a cushion of the physical user interface, leaking typically will result. As discussed above, the cushion is typically formed of a pliable foam. The spacing may be caused by the physical user interface being too loose on the face of the individual. However, in other situations, a user may unevenly tighten a portion of the physical user interface on the face of the user. Thus, a portion of the physical user interface may be tight, while another portion may be loose, resulting in leaking from the loose portion.

In a further method, a second image data associated with at least a portion of the face of the individual after a third period of time is generated. In this method, the second image data is compared to the first image data on one or more characteristics of the face of the individual. The third period of time is at least 2 hours after the second period of time. The purpose of the second image data is to obtain information of the at least a portion of the face of the individual and compare the first and second image data on one or more characteristics of the face of the individual. Such a comparison may indicate a problem with the positioning or the application of the physical user interface. The application of the physical user interface may be problematic such as being too tight on the face of an individual. This comparison may also indicate a problem apart from the physical user interface. For example, a comparison of the second image data and the first image data may indicate problems with the elasticity of the skin or edema issues. This can be used in other methods described above, including the methods 500 and 700.

In yet another method, a further step to the methods 500, 700 includes in response to identifying the at least one facial marking, instructing the user to depress the skin area for causing a temporary indentation. One or more images of the temporary indentation is captured during an elapsed time period in which the skin area bounces back to a full or partial undepressed state. The one or more images for characteristics of skin bounce-back are analyzed. An edema result is determined based on the characteristics of the skin bounce-back. This method may include a further step of instructing the user to depress the skin area with a finger or a probe. This can be used in other methods described above, including the methods 500 and 700.

One or more elements or aspects or steps, or any portion(s) thereof, from one or more of any of claims 1 to 59 below can be combined with one or more elements or aspects or steps, or any portion(s) thereof, from one or more of any of the other claims 1 to 59 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A system comprising:
an electronic interface configured to receive (i) a first real-time video of at least a portion of a face of a user, at least a portion of a head of the user, or a combination of the at least a portion of the face and the head of the user; and (2) a second real-time video of at least a portion of a face of a user, at least a portion of a head of the user, or a combination of at least a portion of the face and the head of the user, the user in the second real-time video wearing a physical user interface;
a memory storing machine-readable instructions; and
a control system including one or more processors configured to execute the machine-readable instructions to:
modifying the first real-time video by superimposing a virtual user interface;
providing feedback to the user in real time on the second real-time video.

2. The system of claim 1, wherein the virtual user interface is a mask and the physical user interface is a mask.

3. The system of claim 2, wherein the mask is a full face mask, a nasal mask, or a nasal pillow mask.

4. The system of claim 1, wherein the feedback is a recommendation to modify the placement of the physical user interface in the second real-time video.

5. The system of claim 4, wherein the feedback identifies the location and type of problem with the positioning of the physical user interface in the second real-time video.

6. The system of claim 1, wherein the feedback is a recommendation to replace the physical user interface in the second real-time video with another physical user interface.

7. The system of claim 1, wherein the feedback is displaying both the first real-time video and the second real-time video in adjacent sections of a screen.

8. The system of claim 1 further including showing the steps of placing the virtual user interface onto the face and the head of the user.

9. The system of claim 1 further including providing an input for the user to select the virtual user interface used in the first real-time video.

10. The system of claim 1, wherein the superimposed virtual user interface moves in sync in the first real-time video with at least a portion of a face of a user, at least a portion of a head of the user, or a combination of the at least a portion of the face and the head of the user.

11. The system of claim 1, wherein superimposing the mask uses a machine learning algorithm that is trained to receive as an input: (i) information from the first real-time video and (2) database from a plurality of virtual user interfaces.

12. The system of claim 11, wherein superimposing the mask uses a machine learning algorithm that is trained to receive as an input further includes: (iii) user-entered information.

13. The system of claim 12, wherein the user-entered information includes (i) measurements of the face or the head; (ii) measurements of one or more features on the face or head; (iii) identification of breathing method; (iv) gender, (v) ethnicity; (vi) amount of hair; or (vii) any combination thereof.

14. The system of claim 1, wherein the first real-time video and the second real-time video are generated using a camera.

15. A method comprising:
displaying a first real-time video of at least a portion of a face of a user, at least a portion of a head of the user, or a combination of the at least a portion of the face and the at least a portion of the head of the user;
modifying the first real-time video by superimposing a virtual user interface;
displaying a second real-time video of at least a portion of a face of a user, at least a portion of a head of the user, or a combination of the at least a portion of the face and the at least a portion of the head of the user, the user in the second real-time video wearing a physical user interface; and
providing feedback to the user in real time on the second real-time video.

16. The method of claim 15, wherein the virtual user interface is a mask and the physical user interface is a mask.

17. (canceled)

18. The method of claim 15, wherein the feedback is a recommendation to modify the placement of the physical user interface in the second real-time video.

19. (canceled)

20. The method of claim 15, wherein the feedback is a recommendation to replace the physical user interface in the second real-time video with another physical user interface.

21-32. (canceled)

33. A method comprising:
generating image data associated with at least a portion of a face of an individual who has disengaged a physical user interface after a first period of time on the face of the individual, the image data being generated within a second period of time;
analyzing the generated image data to determine one or more characteristics of the face of the individual; and
providing feedback to the individual based at least in part on the determined one or more characteristics of the face of the individual.

34. A method comprising:
engaging a physical user interface to a face of an individual;
after a first period of time, disengaging the physical user interface from the face of the individual;
after the disengaging and within a second period of time, generating image data associated with at least a portion of the face of the individual;
analyzing the generated image data to determine one or more characteristics of the face of the individual; and
providing feedback to the individual based at least in part on the determined one or more characteristics of the face of the individual.

35-39. (canceled)
